(19) United States
(12) Patent Application Publication
(10) Pub. No.: US 2021/0002660 A1
(43) Pub. Date: Jan. 7, 2021

(54) ABIOTIC STRESS TOLERANT PLANTS AND METHODS OF USE

(71) Applicants: SINOBIOWAY BIO-AGRICULTURE GROUP CO. LTD., BEIJING (CN); PIONEER OVERSEAS CORPORATION, JOHNSTON, IA (US)

(72) Inventors: GUANGWU CHEN, BEIJING (CN); YANG GAO, BEIJING (CN); GUIHUA LU, SAN DIEGO, CA (US); CHANGGUI WANG, BEIJING (CN); GUOKUI WANG, BEIJING (CN)

(73) Assignees: SINOBIOWAY BIO-AGRICULTURE GROUP CO. LTD., BEIJING (CN); PIONEER OVERSEAS CORPORATION, JOHNSTON, IA (US)

(21) Appl. No.: 16/976,235

(22) PCT Filed: Mar. 5, 2019
(86) PCT No.: PCT/CN2019/076967

§ 371 Date: Aug. 27, 2020

(30) Foreign Application Priority Data
Mar. 6, 2018 (CN) 201810182157.9

(51) Int. Cl. C12N 15/82; C12N 9/02; C12Q 1/6895

(52) U.S. Cl. CPC C12N 15/8261; C12N 15/8271; C12N 15/8273; C12Q 2600/156; C12N 9/0051; C12Q 1/6895; C12Q 2600/13; C12Y 108/07002

(57) ABSTRACT

Isolated polynucleotides and polypeptides, and recombinant DNA constructs are useful for conferring improved drought tolerance and/or nitrogen stress tolerance. Compositions (such as plants or seeds) comprise these recombinant DNA constructs; and methods utilize these recombinant DNA constructs. The recombinant DNA constructs comprise a polynucleotide operably linked to a promoter that is functional in a plant, wherein said polynucleotides encode drought tolerance polypeptide FTR1.

### FIELD

The field of the disclosure relates to plant breeding and genetics and, particularly, relates to recombinant DNA constructs useful in plants for improving tolerance to abiotic stress, such as drought and nitrogen limiting conditions.

### BACKGROUND

Stresses to plants may be caused by both biotic and abiotic agents. For example, biotic causes of stress include infection with pathogen, insect feeding, and parasitism by another plant such as mistletoe. Abiotic stresses include, for example, excessive or insufficient available water, deficiency of nutrient elements adversely, temperature extremes, and synthetic chemicals such as herbicides.

Abiotic stress is the primary cause of crop loss worldwide, causing average yield losses more than 50% for major crops (Boyer, J. S. (1982) Science 218:443-448; Bray, E. A. et al. (2000) In Biochemistry and Molecular Biology of Plants, edited by Buchannan, B. B. et al., Amer. Soc. Plant Biol., pp. 1158-1249). Plants are sessile and must adjust to the prevailing environmental conditions of their surroundings. This has led to their development of a great plasticity in gene regulation, morphogenesis, and metabolism. Adaption and defense strategies involve the activation of genes encoding proteins important in the acclimation or defense towards the different stresses.

Drought is one of the major abiotic stresses that limit crop productivity worldwide, and exposure of plants to a water-limiting environment during various developmental stages appear to activate various physiological and developmental changes. Genetic research has shown that drought tolerance is a quantitative trait, controlled by many genes. Molecular marker-assisted breeding has led to improved drought tolerance in crops. Transgenic approaches to engineering drought tolerance in crops have made progress (Vinocur B. and Altman A. (2005) Curr. Opin. Biotechnol. 16:123-132; Lawlor D W. (2013) J. Exp. Bot. 64:83-108).

The absorption of nitrogen by plants plays an important role during plant growth (Gallais et al., J. Exp. Bot. 55(396):295-306 (2004)). Plants synthesize amino acids from inorganic nitrogen in the environment. Consequently, nitrogen fertilization has been a powerful tool for increasing the yield of cultivated plants, such as rice, maize and soybean. Lack of sufficient plant-available nitrogen for optimum growth and development may be considered as an abiotic stress. In order to avoid pollution by nitrates and to maintain a sufficient profit margin, there is a need to reduce the use of nitrogen fertilizer.

Accordingly, there is a need to develop new compositions and method to improve drought tolerance and/or nitrogen use efficiency. This invention provides such compositions and methods.

### SUMMARY

### The Following Embodiments are Among Those Encompassed by the Disclosure

In one embodiment, the present disclosure includes an isolated polynucleotide, comprising: (a) a polynucleotide with nucleotide sequence of at least 85% sequence identity to SEQ ID NO: 1; (b) a polynucleotide with nucleotide sequence of at least 85% sequence identity to SEQ ID NO: 2; (c) a polynucleotide encoding a polypeptide with amino acid sequence of at least 90% sequence identity to SEQ ID NO: 3; or (d) the full complement of the nucleotide sequence of (a), (b) or (c), wherein an increased expression of the polynucleotide in a plant enhances drought tolerance. In certain embodiments, the isolated polynucleotide comprises the nucleotide sequence of SEQ ID NO: 1 or 2; and the polypeptide comprises the amino acid sequence of SEQ ID NO: 3. In certain embodiments, an increased expression of the polynucleotide in a plant enhances the grain yield under well water conditions or low nitrogen conditions.

The present disclosure also provides a recombinant DNA construct comprising an isolated polynucleotide operably linked to at least one heterologous regulatory element, wherein the polynucleotide comprises: (a) a polynucleotide with nucleotide sequence of at least 85% sequence identity to SEQ ID NO: 1 or 2; (b) a polynucleotide encoding a polypeptide with amino acid sequence of at least 90% sequence identity to SEQ ID NO: 3; or (c) the full complement of the nucleotide sequence of (a) or (b).

Also provided is a modified plant or seed comprising an increased expression of at least one polynucleotide encoding an FTR1 polypeptide compared to a control plant or seed, wherein the polynucleotide comprises: (a) a polynucleotide with nucleotide sequence of at least 85% sequence identity to SEQ ID NO: 1 or 2; (b) a polynucleotide encoding a polypeptide with amino acid sequence of at least 90% sequence identity to SEQ ID NO: 3.

In certain embodiments, the modified plant or seed comprises in its genome a recombinant DNA construct comprising a polynucleotide operably linked to at least one heterologous regulatory element, wherein the polynucleotide comprises: (a) a polynucleotide with nucleotide sequence of at least 85% sequence identity to SEQ ID NO: 1 or 2; (b) a polynucleotide encoding a polypeptide with amino acid sequence of at least 90% sequence identity to SEQ ID NO: 3; or (c) the full complement of the nucleotide sequence of (a) or (b), wherein the said plant exhibits improved drought tolerance and/or improved nitrogen use efficiency when compared to the control plant. In certain embodiments, the improved drought tolerance may be improved seed setting rate or increased yield under drought condition, and the improved nitrogen use efficiency may be increased grain yield under low nitrogen conditions.

In certain embodiments, the modified plant comprises an introduced genetic modification at a genomic locus comprising a polynucleotide encoding a polypeptide with amino acid sequence of at least 90% sequence identity to SEQ ID NO: 3; wherein the introduced genetic modification increases the expression and/or activity of the polypeptide and the plant exhibits improved drought tolerance or nitrogen use efficiency (NUE) when compared to the control plant. In certain embodiments, the genetic modification is introduced into a regulatory element of the polynucleotide

Also provided are methods for increasing drought tolerance in a plant, comprising increasing the expression of at least one polynucleotide encoding a FTR1 polypeptide in the plant compared to a control plant, wherein the polynucleotide comprises: (a) a polynucleotide with nucleotide sequence of at least 85% sequence identity to SEQ ID NO: 1; (b) a polynucleotide with nucleotide sequence of at least 85% sequence identity to SEQ ID NO: 2; and (c) a polynucleotide encoding a polypeptide with amino acid sequence of at least 90% sequence identity to SEQ ID NO: 3.

Also provided are methods for increasing nitrogen use efficiency (NUE) in a plant, comprising increasing the expression of at least one polynucleotide encoding a FTR1 polypeptide in the plant compared to a control plant, wherein the polynucleotide comprises: (a) a polynucleotide with nucleotide sequence of at least 85% identity to SEQ ID NO: 1; (b) a polynucleotide with nucleotide sequence of at least 85% identity to SEQ ID NO: 2; and (c) a polynucleotide encoding a polypeptide with amino acid sequence of at least 90% identity to SEQ ID NO: 3.

In certain embodiments, the expression of the polynucleotide is increased by: (a) increasing the expression of the polynucleotide encoding a FTR1 polypeptide in plant by introducing a recombinant DNA construct into the plant, wherein the recombinant DNA construct comprises the polynucleotide encoding the FTR1 polypeptide operably linked to at least one heterologous regulatory element, wherein the polynucleotide encodes the polypeptide having an amino acid sequence of at least 90% sequence identity compared to SEQ ID NO: 3; or (b) introducing a genetic modification that increases the expression and/or activity of an endogenous polynucleotide encoding the polypeptide having an amino acid sequence of at least 90% sequence identity compared to SEQ ID NO: 3.

Further provided are methods for increasing grain yield in a plant, comprising increasing the expression of at least one polynucleotide encoding a FTR1 polypeptide in the plant compared to a control plant, wherein the polynucleotide comprises: (a) a polynucleotide with nucleotide sequence of at least 85% identity to SEQ ID NO: 1; (b) a polynucleotide with nucleotide sequence of at least 85% identity to SEQ ID NO: 2; and (c) a polynucleotide encoding a polypeptide with amino acid sequence of at least 90% identity to SEQ ID NO: 3.

In certain embodiments, the expression of the polynucleotide is increased by: (a) increasing the expression of the polynucleotide encoding a FTR1 polypeptide in plant by introducing a recombinant DNA construct into the plant, wherein the recombinant DNA construct comprises the polynucleotide encoding the FTR1 polypeptide operably linked to at least one heterologous regulatory element, wherein the polynucleotide encodes the polypeptide having an amino acid sequence of at least 90% sequence identity compared to SEQ ID NO: 3; or (b) introducing a genetic modification that increases the expression and/or activity of an endogenous polynucleotide encoding the polypeptide having an amino acid sequence of at least 90% sequence identity compared to SEQ ID NO: 3.

### BRIEF DESCRIPTION OF THE SEQUENCE LISTING

The disclosure can be more fully understood from the following detailed description and Sequence Listing which form a part of this application.

TABLE 1
Nucleotide and Amino Acid Sequences
Provided in the Sequence Listing

| | Sequence | SEQ ID NO: | SEQ ID NO: |
|---|---|---|---|
| Source species | Description | (Nucleotide) | (Amino Acid) |
| Oryza sativa | OsFTR1 | 1, 2 | 3 |
| Artificial | Primers | 4-5 | n/a |

The Sequence Listing contains the one-letter code for nucleotide sequences and the three-letter code for amino acid sequences as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R. § 1.822.

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants; reference to “a cell” includes one or more cells and equivalents thereof known to those skilled in the art, and so forth.

As used herein:

The term “OsFTR1 (ferredoxin-thioredoxin reductase 1)” refers to a rice polypeptide that confers drought tolerance phenotype and is encoded by the rice gene locus LOC_Os04g44650.1 and any associated allelic variants thereof. “FTR1 polypeptide” refers herein to the OsFTR1 polypeptide and its homologs from other organisms.

The OsFTR1 polypeptide (SEQ ID NO: 3) is encoded by the coding sequence (CDS) (SEQ ID NO: 2) or nucleotide sequence (SEQ ID NO: 1) at rice gene locus LOC_Os04g44650.1 and any associated allelic variants thereof. This polypeptide is annotated as “ferredoxin-thioredoxin reductase, variable chain, putative, expressed” in TIGR (the internet at plant biology msu.edu/index.shtml).

The terms “monocot” and “monocotyledonous plant” are used interchangeably herein. A monocot of the current disclosure includes plants of the Gram ineae family.

The terms “dicot” and “dicotyledonous plant” are used interchangeably herein. A dicot of the current disclosure includes the following families: Brassicaceae, Leguminosae, and Solanaceae.

“Plant” includes reference to whole plants, plant organs, plant tissues, seeds and plant cells and progeny of the same. Plant cells include, without limitation, cells from seeds, suspension cultures, embryos, meristematic regions, callus tissues, leaves, roots, shoots, gametophytes, sporophytes, pollen, and microspores.

“Progeny” comprises any subsequent generation of a plant.

“Modified plant” includes reference to a plant which comprises within its genome a heterologous polynucleotide or modified gene or promoter. For example, the heterologous polynucleotide is stably integrated within the genome such that the polynucleotide is passed on to successive generations. The heterologous polynucleotide may be integrated into the genome alone or as part of a recombinant DNA construct. A T0 plant is directly recovered from the transformation and regeneration process. Progeny of T0 plants are referred to as T1 (first progeny generation), T2 (second progeny generation), etc.

“Heterologous” with respect to sequence means a sequence that originates from a foreign species, or, if from the same species, is substantially modified from its native form in composition and/or genomic locus by deliberate human intervention.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

A “control” or “control plant” or “control plant cell” provides a reference point for measuring changes in phenotype of a subject plant or plant cell in which genetic alteration, such as transformation, has been affected as to a gene of interest. A subject plant or plant cell may be descended from a plant or cell so altered and will comprise the alteration. Typically, the control plant is a wild-type plant or cell, i.e., of the same genotype as the starting material for the genetic alteration which resulted in the subject plant or cell

The term “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar or nitrogen concentrations, or by the observation of the expression level of a gene or genes, or by agricultural observations such as osmotic stress tolerance or yield.

“Agronomic characteristic” is a measurable parameter including but not limited to: greenness, grain yield, growth rate, total biomass or rate of accumulation, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, tiller number, panicle size, early seedling vigor and seedling emergence under low temperature stress.

“Drought” refers to a decrease in water availability to a plant that, especially when prolonged or when occurring during critical growth periods, can cause damage to the plant or prevent its successful growth (e.g., limiting plant growth or seed yield).

“Drought tolerance” reflects a plant's ability to survive under drought conditions without exhibiting substantial physiological or physical deterioration, and/or its ability to recover when water is restored following a period of drought.

“Drought tolerance activity” of a polypeptide indicates that over-expression of the polypeptide in a transgenic plant confers increased drought tolerance of the transgenic plant relative to a reference or control plant.

“Increased drought tolerance” of a plant is measured relative to a reference or control plant, and reflects ability of the plant to survive under drought conditions with less physiological or physical deterioration than a reference or control plant grown under similar drought conditions, or ability of the plant to recover more substantially and/or more quickly than a control plant when water is restored following a period of drought. For example, a plant with “increased drought tolerance” may have a higher grain yield when grown under drought conditions compared to a control plant grown under the same conditions.

“Nitrogen limiting conditions” refers to conditions where the amount of total available nitrogen (e.g., from nitrates, ammonia, or other known sources of nitrogen) is not sufficient to sustain optimal plant growth and development. One skilled in the art would recognize conditions where total available nitrogen is sufficient to sustain optimal plant growth and development. One skilled in the art would recognize what constitutes sufficient amounts of total available nitrogen, and what constitutes soils, media and fertilizer inputs for providing nitrogen to plants. Nitrogen limiting conditions will vary depending upon a few factors, including but not limited to, the particular plant and environmental conditions.

The terms “nitrogen stress tolerance”, “low nitrogen tolerance” and “nitrogen deficiency tolerance” are used interchangeably herein, which indicate a trait of a plant and refer to the ability of the plant to survive under nitrogen limiting conditions or low nitrogen conditions.

“Increased nitrogen stress tolerance” of a polypeptide indicates that over-expression of the polypeptide in a transgenic plant confers increased nitrogen stress tolerance of the transgenic plant relative to a reference or control plant.

“Increased nitrogen stress tolerance” of a plant is measured relative to a reference or control plant, reflects ability of the plant to survive and/or grow better under nitrogen limiting conditions, and means that the nitrogen stress tolerance of the plant is increased by any amount or measured when compared to the nitrogen stress tolerance of the reference or control plant. For example, a plant with “increased nitrogen stress tolerance” may have a higher grain yield when grown under low nitrogen conditions compared to a control plant grown under the same conditions.

“NUE” is nitrogen use efficiency and refers to a plant's ability to utilize nitrogen in low or high levels of fertilizer. It reflects the plant's ability to uptake, assimilate, and/or otherwise utilize nitrogen.

“Paraquat” (1,1-dimethyl-4,4-bipyridinium dichloride), is a foliar-applied and non-selective bipyridinium herbicides, and causes photooxidative stress which further cause damage to plant or prevent its successful growth.

“Paraquat tolerance” is a trait of a plant, reflects the ability to survive and/or grow better when treated with Paraquat solution, compared to a reference or control plant.

“Increased paraquat tolerance” of a plant is measured relative to a reference or control plant, and reflects ability of the plant to survive with less physiological or physical deterioration than a reference or control plant after treated with paraquat solution. In general, tolerance to relative low level of paraquat can be used as a marker of abiotic stress tolerance, such as drought tolerance.

“Oxidative stress” reflects an imbalance between the systemic manifestation of reactive oxygen species and a biological system's ability to readily detoxify the reactive intermediates or to repair the resulting damage. Disturbances in the normal redox state of cells can cause toxic effects through the production of peroxides and free radicals that damage all components of the cell, including proteins, lipids, and DNA.

“Polynucleotide”, “nucleic acid sequence”, “nucleotide sequence”, and “nucleic acid fragment” are used interchangeably and refer to a polymer of RNA or DNA that is single- or double-stranded, optionally containing synthetic, non-natural or altered nucleotide bases. Nucleotides (usually found in their 5′-monophosphate form) are referred to by their single-letter designation as follows: “A” for adenylate or deoxyadenylate, “C” for cytidylate or deoxycytidylate, and “G” for guanylate or deoxyguanylate for RNA or DNA, respectively; “U” for uridylate; “T” for deoxythymidylate; “R” for purines (A or G); “Y” for pyrimidines (C or T); “K” for G or T; “H” for A or C or T; “I” for inosine; and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, and sulfation, gamma-carboxylation of glutamic acid residues, hydroxylation and ADP-ribosylation.

“Isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterogenous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” refers to a combination of nucleic acid fragments that are not normally found together in nature. Accordingly, a recombinant DNA construct may comprise regulatory elements and coding sequences that are derived from different sources, or regulatory elements and coding sequences derived from the same source, but arranged in a manner different than that normally found in nature.

“Regulatory elements” refer to nucleotide sequences located upstream (5′ non-coding sequences), within, or downstream (3′ non-coding sequences) of a coding sequence, and influencing the transcription, RNA processing or stability, or translation of the associated coding sequence. Regulatory elements may include, but are not limited to, promoters, translation leader sequences, introns, and poly-adenylation recognition sequences. The terms “regulatory sequence” and “regulatory element” and “regulatory region” are used interchangeably herein.

“Promoter” refers to a nucleic acid fragment capable of controlling transcription of another nucleic acid fragment.

“Promoter functional in a plant” is a promoter capable of controlling transcription of genes in plant cells whether its origin is from a plant cell or not.

“Tissue-specific promoter” and “tissue-preferred promoter” may refer to a promoter that is expressed predominantly but not necessarily exclusively in one tissue or organ, but that may also be expressed in one specific cell or cell type.

“Developmentally regulated promoter” refers to a promoter whose activity is determined by developmental events.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

“Transformation” as used herein refers to both stable transformation and transient transformation.“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation.“Transient transformation” refers to the introduction of a nucleic acid fragment into the nucleus, or DNA-containing organelle, of a host organism resulting in gene expression without genetically stable inheritance.

An “allele” is one of two or more alternative forms of a gene occupying a given locus on a chromosome. When the alleles present at a given locus on a pair of homologous chromosomes in a diploid plant are the same, that plant is homozygous at that locus. If the alleles present at a given locus on a pair of homologous chromosomes in a diploid plant differ, that plant is heterozygous at that locus. If a transgene is present on one of a pair of homologous chromosomes in a diploid plant, that plant is hemizygous at that locus.

A “gene” refers to a nucleic acid fragment that expresses a functional molecule such as, but not limited to, a specific protein, including regulatory sequences preceding (5′ non-coding sequences) and following (3′ non-coding sequences) the coding sequence. “Native gene” refers to a gene as found in nature with its own regulatory sequences.

A “mutated gene” is a gene that has been altered through human intervention. Such a “mutated gene” has a sequence that differs from the sequence of the corresponding non-mutated gene by at least one nucleotide addition, deletion, or substitution. A mutated plant is a plant comprising a mutated gene.

As “targeted mutation” is a mutation in a native gene that was made by altering a target sequence within the native gene using a method involving a double-strand-break-inducing agent that is capable of inducing a double-strand break in the DNA of the target sequence as disclosed herein of known in the art.

“Genetic modification” refers to a change or alteration in the genomic nucleic acid sequence of a plant introduced by deliberate human activity.

“CRISPR-associated genes” refers to nucleic acid sequences that encode polypeptide components of clustered regularly interspersed short palindromic repeats (CRISPR)-associated systems (Cas), and the genes are generally coupled, associated or close to or in the vicinity of flanking CRISPR loci. The terms “Cas gene”, “CRISPR-associated gene” are used interchangeably herein. Examples include, but are not limited to, Cas3 and Cas9, which encode endonucleases from the CRISPR type I and type II systems, respectively.

“Cas endonuclease” refers to a Cas protein encoded by a Cas gene, wherein said Cas protein is capable of introducing a double strand break into a DNA target sequence. The Cas endonuclease is guided by the guide polynucleotide to recognize and optionally introduce a double strand break at a specific target site into the genome of a cell.

“Guide RNA (gRNA)” refers to a crRNA (CRISPR RNA): tracrRNA fused hybrid RNA molecule encoded by a customizable DNA element that, generally, comprises a copy of a spacer sequence which is complementary to the protospacer sequence of the genomic target site, and a binding domain for an associated-Cas endonuclease of the CRISPR complex.

“Guide polynucleotide” refers to a polynucleotide sequence that can form a complex with a Cas endonuclease and enables the Cas endonuclease to recognize and optionally cleave a DNA target site. The guide polynucleotide can be comprised of a single molecule or a double molecule.

The term “guide polynucleotide/Cas endonuclease system” refers to a complex of a Cas endonuclease and a guide polynucleotide that is capable of introducing a double strand break into a DNA target sequence. The Cas endonuclease unwinds the DNA duplex in close proximity of the genomic target site and cleaves both DNA strands upon recognition of a target sequence by a guide RNA, but only if the correct protospacer-adjacent motif (PAM) is approximately oriented at the 3′ end of the target sequence.

“Genomic target site” refers to a protospacer and a protospacer adjacent motif (PAM) located in a host genome selected for targeted mutation and/or double-strand break.

“Protospacer” refers to a short DNA sequence (12 to 40 bp) that can be targeted for mutation, and/or double-strand break, mediated by enzymatic cleavage with a CRISPR system endonuclease guided by complementary base-pairing with the spacer sequence in the crRNA or sgRNA.

“Protospacer adjacent motif (PAM)” includes a 3 to 8 bp sequence immediately adjacent to the protospacer sequence in the genomic target site.

CRISPR loci (Clustered Regularly Interspaced Short Palindromic Repeats) (also known as SPIDRs-SPacer Interspersed Direct Repeats) constitute a family of recently described DNA loci. CRISPR loci consist of short and highly conserved DNA repeats (typically 24 to 40 bp, repeated from 1 to 140 times—also referred to as CRISPR—repeats) which are partially palindromic. The repeated sequences (usually specific to a species) are interspaced by variable sequences of constant length (typically 20 to 58 bp by depending on the CRISPR locus (WO2007/025097 published Mar. 1, 2007).

Endonucleases are enzymes that cleave the phosphodiester bond within a polynucleotide chain, and include restriction endonucleases that cleave DNA at specific sites without damaging the bases. Restriction endonucleases include Type I, Type II, Type III, and Type IV endonucleases, which further include subtypes. In the Type I and Type III systems, both the methylase and restriction activities are contained in a single complex. Endonucleases also include meganucleases, also known as homing endonucleases (HEases), which like restriction endonucleases, bind and cut at a specific recognition site, however the recognition sites for meganucleases are typically longer, about 18 bp or more (patent application WO-PCT PCT/US12/30061 filed on Mar. 22, 2012). Meganucleases have been classified into four families based on conserved sequence motifs, the families are the LAGLIDADG, GIY-YIG, H—N—H, and His-Cys box families. These motifs participate in the coordination of metal ions and hydrolysis of phosphodiester bonds. HEases are notable for their long recognition sites, and for tolerating some sequence polymorphisms in their DNA substrates.

TAL effector nucleases are a new class of sequence-specific nucleases that can be used to make double-strand breaks at specific target sequences in the genome of a plant or other organism. TAL effector nucleases are created by fusing a native or engineered transcription activator-like (TAL) effector, or functional part thereof, to the catalytic domain of an endonuclease, such as, Foki. The unique, modular TAL effector DNA binding domain allows for the design of proteins with potentially any given DNA recognition specificity (Miller et al. (2011) Nature Biotechnology 29:143-148). Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs consist of an engineered DNA-binding zinc finger domain linked to a non-specific endonuclease domain, for example nuclease domain from a Type IIS endonuclease such as FokI. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3-finger domain recognized a sequence of 9 contiguous nucleotides, with a dimerization requirement of the nuclease, two sets of zinc finger triplets are used to bind an 18 nucleotide recognition sequence.

The terms “target site”, “target sequence”, “target DNA”, “target locus”, “genomic target site”, “genomic target sequence”, and “genomic target locus” are used interchangeably herein and refer to a polynucleotide sequence in the genome (including chloroplastic and mitochondrial DNA) of a plant cell at which a double-strand break is induced in the plant cell genome by a Cas endonuclease. The target site can be an endogenous site in the plant genome, or alternatively, the target site can be heterologous to the plant and thereby not be naturally occurring in the genome, or the target site can be found in a heterologous genomic location compared to where it occurs in nature. As used herein, terms “endogenous target sequence” and “native target sequence” are used interchangeable herein to refer to a target sequence that is endogenous or native to the genome of a plant and is at the endogenous or native position of that target sequence in the genome of the plant.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

“Percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100).

### Turning Now to the Embodiments

Embodiments include isolated polynucleotides and polypeptides, and recombinant DNA constructs useful for conferring drought tolerance and/or nitrogen stress tolerance; compositions (such as plants or seeds) comprising these recombinant DNA constructs; and methods utilizing these recombinant DNA constructs.

Isolated Polynucleotides and Polypeptides:

The present disclosure includes the following isolated polynucleotides and polypeptides:

An isolated polynucleotide comprising: (i) a nucleic acid sequence encoding a polypeptide having at least 80% (e.g., 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO: 3; or (ii) a full complement of the nucleic acid sequence of (i). In certain embodiments, an increased expression of the encoded polypeptide increases plant drought tolerance, and/or paraquat tolerance activity, and/or low nitrogen tolerance activity. In certain embodiments, an increased expression of the encoded polypeptide increases plant yield under normal conditions.

An isolated polypeptide having an amino acid sequence of at least 80% (e.g., 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 3. In certain embodiment, an increased expression of the polypeptide increases plant drought tolerance and/or paraquat tolerance activity and/or low nitrogen tolerance activity. In certain embodiments, an increased expression of the encoded polypeptide increases plant grain yield under normal conditions.

An isolated polynucleotide comprising (i) a nucleic acid sequence of at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO: 2; (ii) a nucleic acid sequence of at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO: 1; or (iii) a full complement of the nucleic acid sequence of (i) or (ii). In certain embodiments, an increased expression of the polypeptide improves plant drought tolerance and/or paraquat tolerance activity and/or low nitrogen tolerance activity. In certain embodiments, an increased expression of the encoded polypeptide increases plant grain yield under normal conditions.

It is understood, as those skilled in the art will appreciate, that the disclosure encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

Recombinant DNA Constructs:

In one aspect, the present disclosure includes recombinant DNA constructs.

In one embodiment, the recombinant DNA construct comprises a polynucleotide operably linked to at least one regulatory element (e.g., a promoter functional in a plant), wherein the polynucleotide comprises (i) a nucleic acid sequence encoding an amino acid sequence of at least 80% (e.g., 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO: 3; or (ii) a full complement of the nucleic acid sequence of (i).

In another embodiment, the recombinant DNA construct comprises a polynucleotide operably linked to at least one regulatory element (e.g., a promoter functional in a plant), wherein said polynucleotide comprises (i) a nucleic acid sequence of at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO: 1 or 2; or (ii) a full complement of the nucleic acid sequence of (i).

Regulatory Elements:

The recombinant DNA construct of the present disclosure may comprise at least one regulatory element.

In certain embodiments, the regulatory element is a promoter or enhancer.

A number of promoters can be used in recombinant DNA constructs of the present disclosure. The promoters can be selected based on the desired outcome, and may include constitutive, tissue-specific, inducible, or other promoters for expression in the host organism.

Suitable constitutive promoters for use in a plant host cell include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611.

A tissue-specific or developmentally-regulated promoter is a DNA sequence which regulates the expression of a DNA sequence selectively in the cells/tissues of a plant, such as in those cells/tissues critical to tassel development, seed set, or both, and which usually limits the expression of such a DNA sequence to the developmental period of interest (e.g. tassel development or seed maturation) in the plant. Any identifiable promoter which causes the desired temporal and spatial expression may be used in the methods of the present disclosure.

Many leaf-preferred promoters are known in the art (Yamamoto et al. (1997) Plant J. 12(2):255-265; Kwon et al. (1994) Plant Physiol. 105:357-367; Yamamoto et al. (1994) Plant Cell Physiol. 35(5):773-778; Gotor et al. (1993) Plant J. 3:509-518; Orozco et al. (1993) Plant Mol. Biol. 23(6):1129-1138; and Matsuoka et al. (1993) Proc. Natl. Acad. Sci. USA 90(20):9586-9590).

Promoters which are seed or embryo-specific and may be useful in the disclosure include soybean Kunitz trypsin inhibitor (Kti3, Jofuku and Goldberg. (1989) Plant Cell 1:1079-1093), convicilin, vicilin, and legumin (pea cotyledons) (Rerie, W. G., et al. (1991) Mol. Gen. Genet. 259:149-157; Newbigin, E. J., et al. (1990) Planta 180:461-470; Higgins, T. J. V., et al. (1988) Plant. Mol. Biol. 11:683-695), zein (maize endosperm) (Schemthaner, J. P., et al. (1988) EMBO J. 7:1249-1255), phaseolin (bean cotyledon) (Segupta-Gopalan, C., et al. (1985) Proc. Natl. Acad. Sci. 82:3320-3324), phytohemagglutinin (bean cotyledon) (Voelker, T. et al. (1987) EMBO J. 6:3571-3577), B-conglycinin and glycinin (soybean cotyledon) (Chen, Z-L, et al. (1988) EMBO J. 7:297-302), glutelin (rice endosperm), hordein (barley endosperm) (Marris, C., et al. (1988) Plant Mol. Biol. 10:359-366), glutenin and gliadin (wheat endosperm) (Colot, V., et al. (1987) EMBO J. 6:3559-3564). Promoters of seed-specific genes operably linked to heterologous coding regions in chimeric gene constructions maintain their temporal and spatial expression pattern in transgenic plants. Such examples include Arabidopsis 2S seed storage protein gene promoter to express enkephalin peptides in Arabidopsis and Brassica napus seeds (Vanderkerckhove et al. (1989) Bio/Technology 7: L929-932), bean lectin and bean beta-phaseolin promoters to express luciferase (Riggs et al. (1989) Plant Sci. 63:47-57), and wheat glutenin promoters to express chloramphenicol acetyl transferase (Colot et al. (1987) EMBO J 6:3559-3564).

Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

Promoters for use in certain embodiments include the following: 1) the stress-inducible promoter RD29A (Kasuga et al. (1999) Nature Biotechnol. 17:287-291); 2) the stress-inducible promoter Rab17 (Vilardell et al. (1991) Plant Mol. Bio. 17:985-993; Kamp Busk et al. (1997) Plant J 11(6):1285-1295); 3) the barley promoter B22E whose expression is specific to the pedicel in developing maize kernels (“Primary Structure of a Novel Barley Gene Differentially Expressed in Immature Aleurone Layers”. Klemsdal, S. S. et al. (1991) Mol. Gen. Genet. 228(1/2):9-16); and 4) maize promoter Zag2 (“Identification and molecular characterization of ZAG1, the maize homolog of the Arabidopsis floral homeotic gene AGAMOUS”, Schmidt, R. J. et al. (1993) Plant Cell 5(7):729-737; “Structural characterization, chromosomal localization and phylogenetic evaluation of two pairs of AGAMOUS-like MADS-box genes from maize”, Theissen et al. (1995) Gene 156(2):155-166; NCBI GenBank Accession No. X80206)). Zag2 transcripts can be detected 5 days prior to pollination to 7 to 8 days after pollination (“DAP”), and directs expression in the carpel of developing female inflorescences and CimI which is specific to the nucleus of developing maize kernels. CimI transcript is detected 4 to 5 days before pollination to 6 to 8 DAP. Other useful promoters include any promoter which can be derived from a gene whose expression is maternally associated with developing female florets.

For the expression of a polynucleotide in developing seed tissue, promoters of particular interest include seed-preferred promoters, particularly early kernel/embryo promoters and late kernel/embryo promoters. Kernel development post-pollination is divided into approximately three primary phases. The lag phase of kernel growth occurs from about 0 to 10-12 DAP. During this phase the kernel is not growing significantly in mass, but rather important events are being carried out that will determine kernel vitality (e.g., number of cells established). The linear grain fill stage begins at about 10-12 DAP and continues to about 40 DAP. During this stage of kernel development, the kernel attains almost all of its final mass, and various storage products (i.e., starch, protein, oil) are produced. Finally, the maturation phase occurs from about 40 DAP to harvest. During this phase of kernel development, the kernel becomes quiescent and begins to dry down in preparation for a long period of dormancy prior to germination. As defined herein “early kernel/embryo promoters” are promoters that drive expression principally in developing seed during the lag phase of development (i.e., from about 0 to about 12 DAP). “Late kernel/embryo promoters”, as defined herein, drive expression principally in developing seed from about 12 DAP through maturation. There may be some overlap in the window of expression. The choice of the promoter will depend on the ABA-associated sequence utilized and the phenotype desired.

Early kernel/embryo promoters include, for example, Cim1 that is active 5 DAP in particular tissues (WO 00/11177), which is herein incorporated by reference. Other early kernel/embryo promoters include the seed-preferred promoters end1 which is active 7-10 DAP, and end2, which is active 9-14 DAP in the whole kernel and active 10 DAP in the endosperm and pericarp (WO 00/12733), herein incorporated by reference. Additional early kernel/embryo promoters that find use in certain methods of the present disclosure include the seed-preferred promoter Itp2 (U.S. Pat. No. 5,525,716); maize Zm40 promoter (U.S. Pat. No. 6,403,862); maize nuc1c (U.S. Pat. No. 6,407,315); maize ckx1-2 promoter (U.S. Pat. No. 6,921,815 and US Patent Application Publication Number 2006/0037103); maize lec1 promoter (U.S. Pat. No. 7,122,658); maize ESR promoter (U.S. Pat. No. 7,276,596); maize ZAP promoter (U.S. Patent Application Publication Numbers 20040025206 and 20070136891); maize promoter eep1 (U.S. Patent Application Publication Number 20070169226); and maize promoter ADF4 (U.S. Patent Application No. 60/963,878, filed 7 Aug. 2007).

Additional promoters for regulating the expression of the nucleotide sequences of the present disclosure in plants are stalk-specific promoters, including the alfalfa S2A promoter (GenBank Accession No. EF030816; Abrahams et al. (1995) Plant Mol. Biol. 27:513-528) and S2B promoter (GenBank Accession No. EF030817) and the like, herein incorporated by reference.

Promoters may be derived in their entirety from a native gene, or be composed of different elements derived from different promoters found in nature, or even comprise synthetic DNA segments.

Promoters for use in certain embodiments of the current disclosure may include: RIP2, mLIP15, ZmCOR1, Rab17, CaMV 35S, RD29A, B22E, Zag2, SAM synthetase, ubiquitin, CaMV 19S, nos, Adh, sucrose synthase, R-allele, the vascular tissue preferred promoters S2A (Genbank accession number EF030816) and S2B (Genbank accession number EF030817), and the constitutive promoter GOS2 from Zea mays; root preferred promoters, such as the maize NAS2 promoter, the maize Cyclo promoter (US 2006/0156439, published Jul. 13, 2006), the maize ROOTMET2 promoter (WO05063998, published Jul. 14, 2005), the CR1B10 promoter (WO06055487, published May 26, 2006), the CRWAQ81 (WO05035770, published Apr. 21, 2005) and the maize ZRP2.47 promoter (NCBI accession number: U38790; GI No. 1063664).

Recombinant DNA constructs of the present disclosure may also include other regulatory elements, including but not limited to, translation leader sequences, introns, and polyadenylation recognition sequences. In certain embodiments, a recombinant DNA construct further comprises an enhancer or silencer.

An intron sequence can be added to the 5′ untranslated region, the protein-coding region or the 3′ untranslated region to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg. (1988) Mol. Cell Biol. 8:4395-4405; Callis et al. (1987) Genes Dev. 1:1183-1200).

Compositions:

A composition of the present disclosure is a plant comprising in its genome any of the recombinant DNA constructs of the present disclosure (such as any of the constructs discussed above). Compositions also include any progeny of the plant, and any seed obtained from the plant or its progeny, wherein the progeny or seed comprises within its genome the recombinant DNA construct. Progeny includes subsequent generations obtained by self-pollination or out-crossing of a plant. Progeny also includes hybrids and inbreds.

The plant may be a monocotyledonous or dicotyledonous plant, for example, a rice or maize or soybean plant, such as a maize hybrid plant or a maize inbred plant. The plant may also be sunflower, sorghum, canola, wheat, alfalfa, cotton, barley, millet, sugar cane or switchgrass.

The recombinant DNA construct may be stably integrated into the genome of the plant.

Particular embodiments include but are not limited to the following:

1. A transgenic plant (for example, a rice or maize or soybean plant) comprising in its genome a recombinant DNA construct comprising a polynucleotide operably linked to at least one regulatory element, wherein said polynucleotide encodes a polypeptide having an amino acid sequence of at least 80% (e.g., 81%, 82%, 83%, 84%, 85%, 86%, 87%, 88%, 89%, 90%, 91%, 92%, 93%, 94%, 95%, 96%, 97%, 98%, 99%, or 100%) sequence identity, to SEQ ID NO: 3, and wherein said plant exhibits increased drought tolerance and/or paraquat tolerance and/or nitrogen stress tolerance when compared to a control plant. The plant may further exhibit an alteration of at least one agronomic characteristic when compared to the control plant.

2. A transgenic plant (for example, a rice or maize or soybean plant) comprising in its genome a targeted genetic modification at a genomic locus comprising a polynucleotide encoding a polypeptide having an amino acid sequence of at least 80% (e.g., 81%, 82%, 83%, 84%, 85%, 86%, 87%, 88%, 89%, 90%, 91%, 92%, 93%, 94%, 95%, 96%, 97%, 98%, 99%, or 100%) sequence identity, to SEQ ID NO: 3, wherein the introduced genetic modification increases the expression and/or activity of the polypeptide and said plant exhibits increased drought tolerance and/or paraquat tolerance and/or nitrogen stress tolerance when compared to a control plant.

3. Any progeny of the above plants in embodiment 1-2, any seeds of the above plants in embodiment 1-2, any seeds of progeny of the above plants in embodiment 1-2, and cells from any of the above plants in embodiment 1-2 and progeny thereof.

In certain embodiments, drought tolerance is evaluated by the ability of a plant to maintain sufficient yield (at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% yield) in field testing under simulated or naturally-occurring drought conditions (e.g., by measuring for substantially equivalent yield under drought conditions compared to non-drought conditions, or by measuring for less yield loss under drought conditions compared to yield loss exhibited by a control or reference plant).

In certain embodiments, nitrogen stress tolerance is evaluated by the ability of a plant to maintain sufficient yield (at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% yield) in field testing under simulated or naturally-occurring low nitrogen conditions (e.g., by measuring for substantially equivalent yield under low conditions compared to normal nitrogen conditions, or by measuring for less yield loss under low nitrogen conditions compared to a control or reference plant).

Parameters such as recovery degree, survival rate, paraquat tolerance rate, gene expression level, water use efficiency, level or activity of an encoded protein, and others are typically presented with reference to a control cell or control plant.

One of ordinary skill in the art would readily recognize a suitable control or reference plant to be utilized when assessing or measuring an agronomic characteristic or phenotype of a transgenic plant using compositions or methods as described herein.

Methods:

Provided is a method for producing a transgenic plant comprising transforming a plant cell with any of the isolated polynucleotides or recombinant DNA constructs of the present disclosure and regenerating a transgenic plant from the transformed plant cell, wherein, the transgenic plant and the transgenic seed obtained by this method may be used in other methods of the present disclosure.

Also provided is a method for altering the level of expression of a polypeptide of the disclosure in a host cell comprising: (a) transforming a host cell with a recombinant DNA construct of the present disclosure; and (b) growing the transformed host cell under conditions that are suitable for the expression of the recombinant DNA construct, wherein the expression of the recombinant DNA construct results in production of altered levels of the polypeptide of the disclosure in the transformed host cell.

Further provided is a method of increasing drought tolerance and/or paraquat tolerance and/or nitrogen stress tolerance in a plant, comprising introducing a targeted genetic modification at a genomic locus comprising a polynucleotide encoding a polypeptide with amino acid sequence of at least 90% sequence identity to SEQ ID NO: 3, thereby increasing the level and/or activity of the encoded polypeptide.

Also provided is method of increasing drought tolerance and/or paraquat tolerance and/or nitrogen stress tolerance in a plant, comprising: (a) introducing into a regenerable plant cell a recombinant DNA construct comprising a polynucleotide operably linked to at least one regulatory element (for example, a promoter functional in a plant), wherein the polynucleotide encodes a polypeptide having an amino acid sequence of at least 90%, 91%, 92%, 93%, 94%, 95%, 96%, 97%, 98%, 99% or 100% sequence identity to SEQ ID NO: 3; (b) regenerating a transgenic plant from the regenerable plant cell after step (a), wherein the transgenic plant comprises in its genome the recombinant DNA construct and exhibits increased drought tolerance and/or paraquat tolerance and/or nitrogen stress tolerance when compared to a control plant; and further (c) obtaining a progeny plant derived from transgenic plant, wherein said progeny plant comprises in its genome the recombinant DNA construct and exhibits increased drought tolerance and/or paraquat tolerance and/or nitrogen stress tolerance when compared to a control plant.

Also provided is a method of evaluating drought tolerance and/or paraquat tolerance and/or nitrogen stress tolerance in a plant comprising (a) obtaining a transgenic plant, which comprises in its genome a recombinant DNA construct comprising a polynucleotide operably linked to at least one regulatory element (for example, a promoter functional in a plant), wherein said polynucleotide encodes a polypeptide having an amino acid sequence of at least 90%, 91%, 92%, 93%, 94%, 95%, 96%, 97%, 98%, 99%, or 100% sequence identity, to SEQ ID NO: 3; (b) obtaining a progeny plant derived from said transgenic plant, wherein the progeny plant comprises in its genome the recombinant DNA construct; and (c) evaluating the progeny plant for drought tolerance and/or paraquat tolerance and/or nitrogen stress tolerance compared to a control plant.

Also provided is a method of determining an alteration of an agronomic characteristic in a plant comprising (a) obtaining a transgenic plant which comprises in its genome a recombinant DNA construct comprising a polynucleotide operably linked to at least one regulatory element (for example, a promoter functional in a plant), wherein said polynucleotide encodes a polypeptide having an amino acid sequence of at least 90%, 91%, 92%, 93%, 94%, 95%, 96%, 97%, 98%, 99% or 100% sequence identity when compared to SEQ ID NO: 3; (b) obtaining a progeny plant derived from said transgenic plant, wherein the progeny plant comprises in its genome the recombinant DNA construct; and (c) determining whether the progeny plant exhibits an alteration in at least one agronomic characteristic when compared, optionally under water limiting conditions to a control plant.

In any of the preceding methods or any other embodiments of methods of the present disclosure, in said introducing step, the said regenerable plant cell may comprise a callus cell, an embryogenic callus cell, a gametic cell, a meristematic cell, or a cell of an immature embryo. The regenerable plant cells may derive from an inbred maize plant.

In any of the preceding methods or any other embodiments of methods of the present disclosure, said regenerating step may comprise the following: (i) culturing said transformed plant cells in a medium comprising an embryogenic promoting hormone until callus organization is observed; (ii) transferring said transformed plant cells of step (i) to a first media which includes a tissue organization promoting hormone; and (iii) subculturing said transformed plant cells after step (ii) onto a second media, to allow for shoot elongation, root development or both.

In any of the preceding methods or any other embodiments of methods of the present disclosure, the step of determining an alteration of an agronomic characteristic in a transgenic plant, if applicable, may comprise determining whether the transgenic plant exhibits an alteration of at least one agronomic characteristic when compared, under varying environmental conditions, to a control plant not comprising the recombinant DNA construct.

In any of the preceding methods or any other embodiments of methods of the present disclosure, the step of determining an alteration of an agronomic characteristic in a progeny plant, if applicable, may comprise determining whether the progeny plant exhibits an alteration of at least one agronomic characteristic when compared, under varying environmental conditions, to a control plant not comprising the recombinant DNA construct.

In any of the preceding methods or any other embodiments of methods of the present disclosure, the plant may exhibit the alteration of at least one agronomic characteristic when compared, under water limiting conditions to a control plant.

In any of the preceding methods or any other embodiments of methods of the present disclosure, alternatives exist for introducing into a regenerable plant cell a recombinant DNA construct comprising a polynucleotide operably linked to at least one regulatory element. For example, one may introduce into a regenerable plant cell a regulatory element (such as one or more enhancers, optionally as part of a transposable element), and then screen for an event in which the regulatory element is operably linked to an endogenous gene encoding a polypeptide of the instant disclosure.

The introduction of recombinant DNA constructs of the present disclosure into plants may be carried out by any suitable technique, including but not limited to direct DNA uptake, chemical treatment, electroporation, microinjection, cell fusion, infection, vector-mediated DNA transfer, bombardment, or Agrobacterium-mediated transformation. Techniques for plant transformation and regeneration have been described in International Patent Publication WO 2009/006276, the contents of which are herein incorporated by reference.

In addition, methods to modify or alter the host endogenous genomic DNA are available. This includes altering the host native DNA sequence or a pre-existing transgenic sequence including regulatory elements, coding and non-coding sequences. These methods are also useful in targeting nucleic acids to pre-engineered target recognition sequences in the genome. As an example, the genetically modified cell or plant described herein, is generated using “custom” engineered endonucleases such as meganucleases produced to modify plant genomes (e.g., WO 2009/114321; Gao et al. (2010) Plant Journal 1:176-187). Another site-directed engineering is through the use of zinc finger domain recognition coupled with the restriction properties of restriction enzyme (e.g., Urnov, et al. (2010) Nat Rev Genet. 11(9):636-46; Shukla, et al. (2009) Nature 459 (7245):437-41). A transcription activator-like (TAL) effector-DNA modifying enzyme (TALE or TALEN) is also used to engineer changes in plant genome. See e.g., US20110145940, Cermak et al., (2011) Nucleic Acids Res. 39(12) and Boch et al., (2009), Science 326 (5959): 1509-12. Site-specific modification of plant genomes can also be performed using the bacterial type II CRISPR (clustered regularly interspaced short palindromic repeats)/Cas (CRISPR-associated) system. See e.g., Belhaj et al., (2013), Plant Methods 9: 39; The CRISPR/Cas system allows targeted cleavage of genomic DNA guided by a customizable small noncoding RNA.

The development or regeneration of plants containing the foreign, exogenous isolated nucleic acid fragment that encodes a protein of interest is well known in the art. The regenerated plants may be self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant containing a desired polypeptide is cultivated using methods well known to one skilled in the art.

Stacking of Traits in Transgenic Plant

Transgenic plants may comprise a stack of one or more drought tolerance polynucleotides disclosed herein with one or more additional polynucleotides resulting in the production or suppression of multiple polypeptide sequences. Transgenic plants comprising stacks of polynucleotide sequences can be obtained by either or both of traditional breeding methods or through genetic engineering methods. These methods include, but are not limited to, breeding individual lines each comprising a polynucleotide of interest, transforming a transgenic plant comprising a gene disclosed herein with a subsequent gene and cotransformation of genes into a single plant cell. As used herein, the term “stacked” includes having the multiple traits present in the same plant (i.e., both traits are incorporated into the nuclear genome, one trait is incorporated into the nuclear genome and one trait is incorporated into the genome of a plastid or both traits are incorporated into the genome of a plastid). In one non-limiting example, “stacked traits” comprise a molecular stack where the sequences are physically adjacent to each other. A trait, as used herein, refers to the phenotype derived from a particular sequence or groups of sequences. Co-transformation of genes can be carried out using single transformation vectors comprising multiple genes or genes carried separately on multiple vectors. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or over-expression cassettes to generate the desired combination of traits in the plant. It is further recognized that polynucleotide sequences can be stacked at a desired genomic location using a site-specific recombination system. See, for example, WO 1999/25821, WO 1999/25854, WO 1999/25840, WO 1999/25855 and WO 1999/25853, all of which are herein incorporated by reference.

### EXAMPLES

The present disclosure is further illustrated in the following examples, in which parts and percentages are by weight and degrees are Celsius, unless otherwise stated. It should be understood that these examples, while indicating embodiments of the disclosure, are given by way of illustration only. From the above discussion and these examples, one skilled in the art can ascertain the essential characteristics of this disclosure, and without departing from the spirit and scope thereof, can make various changes and modifications of the disclosure to adapt it to various usages and conditions. Furthermore, various modifications of the disclosure in addition to those shown and described herein will be apparent to those skilled in the art from the foregoing description. Such modifications are also intended to fall within the scope of the appended claims.

### Example 1

### Cloning and Vector Construction of Drought Tolerance Gene

Based on our preliminary screening of rice activation tagging population and the sequence information of gene ID LOC_Os04g44650.1, the OsFTR1 cDNA was cloned from pooled cDNA from leaf, stem and root tissues of Zhonghua 11 plant using conventional methods and the following primers.

gc-5898: 5′-CAATCGGCATCTCTTATCCTCAC-3′

gc-5899: 5′-GACGACGAGAGCACATTATTCTG-3′

The PCR amplified product which is 577 bp in length was extracted after the agarose gel electrophoresis using a column kit and then ligated with TA cloning vectors. The sequence and orientation in the construct were confirmed by sequencing. Then the gene was cloned into plant binary construct DP0158 (pCAMBIA1300-DsRed). The cloned nucleotide sequence in construct of DP0691 and coding sequence of OsFTR1 are provided as SEQ ID NO: 1 and 2, the encoded amino acid sequence of OsFTR1 is shown in SEQ ID NO: 3.

### Example 2

### Transformation to Get Transgenic Rice Lines

The vector prepared in Example 1 or an empty vector (DP0158) were transformed into Zhonghua 11 (Oryza sativa L.) by the Agrobacteria-mediated method as described by Lin and Zhang ((2005) Plant Cell Rep. 23:540-547). Zhonghua 11 was cultivated by the Institute of Crop Sciences, Chinese Academy of Agricultural Sciences. The first batch of seeds used in this research was provided by Beijing Weiming Kaituo Agriculture Biotech Co., Ltd. Calli induced from embryos was transformed with Agrobacteria with the vector. Transgenic seedlings (T0) generated in the transformation laboratory were transplanted in the field to get T1 seeds. The T1 and T2 seeds were stored at cold room (4° C.). The expression vectors contain marker genes. T1 and T2 seeds which showed red color under green fluorescent light were transgenic seeds and were used in the following trait screening.

### Example 3

### Field Drought Assays of Mature Transgenic Rice Plants

For the field drought assays of mature rice plants, 12 transgenic lines from each gene construct were tested. The T2 seeds were first sterilized, and the germinated seeds were planted in a seedbed field. At 3-leaf stage, the seedlings were transplanted into the testing field with 4 replicates and 10 plants per replicate for each transgenic line, and the 4 replicates were planted in the same block. ZH11-TC and DP0158 seedlings were nearby the transgenic lines in the same block, and were used as controls in the statistical analysis.

The rice plants were managed by normal practice using pesticides and fertilizers. Watering was stopped at the panicle initiation stage, to give drought stress at flowering stage depending on the weather conditions (temperature and humidity). The soil water content was measured every 4 days at about 10 sites per block using TDR30 (Spectrum Technologies, Inc.).

Plant phenotypes were observed and recorded during the experiments. The phenotypes include heading date, leaf rolling degree, drought sensitivity and drought tolerance. Special attention was paid to leaf rolling degree at noontime. At the end of the growing season, six representative plants of each transgenic line were harvested from the middle of the row per line, and grain yield per plant was measured. The grain yield data were statistically analyzed using mixed linear model. Positive transgenic lines were selected based on the analysis (P<0.1).

### Field DRT Validation Results of OsFTR1 (DP0691) Transgenic Rice

The soil volumetric water content decreased from 40% to 10% during panicle heading stage. 19 days after stopping watering, the main stem panicles reached panicle initiation stage VIII, and the tiller panicles reached panicle initiation stage VI-VIE, and the rice plants began to show leaf roll phenotype. 33 days after stopping watering, 50% panicles headed out. Six OsFTR1 transgenic lines DP0691.01, DP0691.04, DP0691.07, DP0691.11, DP0691.12, and DP0691.13 showed better seed setting at the maturation stage.

The grain yield analysis showed that the grain yield per plant of OsFTR1 transgenic rice was greater than ZH11-TC and DP0158 controls at the construct level. Four OsFTR1 transgenic lines showed significantly greater grain yields per plants than ZH11-TC plants, and two OsFTR1 transgenic lines showed significantly greater grain yields per plants than DP0158 control plants (Table 2). These results indicate that increased expression of OsFTR1 increased drought tolerance and increased the grain yield per plant after drought stress at flowering stage.

TABLE 2
Grain yield analysis of OsFTR1 transgenic rice plants
under field drought conditions (1^{st} experiment)

| | Number of | Number of | | | | | | | |
|---|---|---|---|---|---|---|---|---|---|
| | survival | harvested | Yield per | CK = ZH11-TC | | | CK = DP0158 | | |
| Line ID | plants | plants | plant (g) | Diff | P value | P ≤ 0.1 | Diff | P value | P ≤ 0.1 |
| DP0691 | | | 8.83 | 1.76 | 0.114 | | 1.05 | 0.345 | |
| (Construct) | | | | | | | | | |
| ZH11-TC | 40 | 24 | 7.07 | | | | | | |
| DP0158 | 40 | 24 | 7.78 | | | | | | |
| DP0691.01 | 40 | 24 | 11.61 | 4.54 | 0.000 | Y | 3.84 | 0.003 | Y |
| DP0691.02 | 40 | 22 | 6.96 | −0.11 | 0.933 | | −0.82 | 0.523 | |
| DP0691.03 | 40 | 24 | 8.33 | 1.26 | 0.331 | | 0.55 | 0.665 | |
| DP0691.04 | 40 | 24 | 8.92 | 1.85 | 0.153 | | 1.15 | 0.377 | |
| DP0691.07 | 40 | 24 | 10.27 | 3.20 | 0.014 | Y | 2.49 | 0.056 | Y |
| DP0691.08 | 40 | 24 | 8.80 | 1.73 | 0.175 | | 1.02 | 0.431 | |
| DP0691.09 | 40 | 24 | 6.86 | −0.21 | 0.869 | | −0.92 | 0.479 | |
| DP0691.10 | 37 | 24 | 7.94 | 0.86 | 0.505 | | 0.16 | 0.903 | |
| DP0691.11 | 40 | 24 | 9.56 | 2.49 | 0.054 | Y | 1.78 | 0.154 | |
| DP0691.12 | 40 | 24 | 9.45 | 2.38 | 0.062 | Y | 1.67 | 0.200 | |
| DP0691.13 | 39 | 24 | 8.36 | 1.28 | 0.313 | | 0.58 | 0.650 | |
| DP0691.15 | 40 | 24 | 8.88 | 1.80 | 0.164 | | 1.10 | 0.399 | |

The same 12 OsFTR1 transgenic rice plants were tested again. Watering was stopped when the main stem panicles reached panicle initiation stage IV. 21 days after stopping watering, 50% panicles headed out, and the rice plants started to show drought stress phenotype. The soil volumetric water content decreased from 35% to 8% during heading and maturation stage.

Grain yield analysis showed that OsFTR1 transgenic rice plants exhibited significantly greater grain yield per plant than ZH11-TC and DP0158 controls at the construct level. Five OsFTR1 transgenic lines exhibited significantly greater grain yields per plant than ZH11-TC and DP0158 controls at the line level (Table 3). These results indicate that OsFTR1 transgenic rice plant gained drought tolerance and exhibited greater grain yield increase per plant.

TABLE 3
Grain yield analysis of OsFTR1 transgenic rice plants
under field drought conditions (2^{nd} experiment)

| | Number of | Number of | | | | | | | |
|---|---|---|---|---|---|---|---|---|---|
| | survival | harvested | Yield per | ZH11-TC | | | DP0158 | | |
| Line ID | plants | plants | plant (g) | Diff | P value | P ≤ 0.1 | Diff | P value | P ≤ 0.1 |
| DP0691 | | | 5.91 | 1.66 | 0.058 | Y | 1.78 | 0.041 | Y |
| (Construct) | | | | | | | | | |
| ZH11-TC | 40 | 24 | 4.25 | | | | | | |
| DP0158 | 40 | 24 | 4.13 | | | | | | |
| DP0691.01 | 40 | 24 | 6.95 | 2.70 | 0.003 | Y | 2.81 | 0.002 | Y |
| DP0691.02 | 40 | 24 | 4.32 | 0.06 | 0.942 | | 0.18 | 0.839 | |
| DP0691.03 | 40 | 24 | 3.92 | −0.33 | 0.710 | | −0.21 | 0.810 | |
| DP0691.04 | 40 | 24 | 4.45 | 0.20 | 0.826 | | 0.32 | 0.726 | |
| DP0691.07 | 40 | 24 | 8.64 | 4.38 | 0.000 | Y | 4.50 | 0.000 | Y |
| DP0691.08 | 40 | 24 | 7.68 | 3.43 | 0.000 | Y | 3.55 | 0.000 | Y |
| DP0691.09 | 38 | 24 | 4.52 | 0.27 | 0.766 | | 0.39 | 0.673 | |
| DP0691.10 | 40 | 24 | 4.52 | 0.27 | 0.769 | | 0.39 | 0.667 | |
| DP0691.11 | 40 | 24 | 7.44 | 3.19 | 0.000 | Y | 3.31 | 0.000 | Y |
| DP0691.12 | 40 | 24 | 5.43 | 1.18 | 0.186 | | 1.30 | 0.146 | |
| DP0691.13 | 40 | 24 | 4.91 | 0.66 | 0.472 | | 0.78 | 0.385 | |
| DP0691.15 | 40 | 24 | 8.19 | 3.93 | 0.000 | Y | 4.05 | 0.000 | Y |

### Example 4

### Laboratory Paraquat Assay of Transgenic Rice Plants

Paraquat (1,1-dimethyl-4,4-bipyridinium dichloride), is a foliar-applied and non-selective bipyridinium herbicide, and it is one of the most widely used herbicides in the world, controlling weeds in a huge variety of crops like corn, rice, soybean etc. In plant cells, paraquat mainly targets chloroplasts by accepting electrons from photosystem I and then reacting with oxygen to produce superoxide and hydrogen peroxide, which cause photooxidative stress. Drought stress usually leads to increased reactive oxygen species (ROS) in plants and sometimes, the drought tolerance of plant is associated with enhanced antioxidative ability. Paraquat is a potent oxidative stress inducer; it greatly increases the ROS production and inhibits the regeneration of reducing equivalents and compounds necessary for the activity of the antioxidant system. The ROS generation is enhanced under abiotic stress conditions, and the plant responses range from tolerance to death depending on the stress intensity and its associated-ROS levels. Relative low level of paraquat can mimic the stress-associated ROS production and used as a stress tolerance marker in plant stress biology (Hasaneen M. N. A. (2012) Herbicide-Properties, Synthesis and Control of Weeds book). Therefore, the paraquat tolerance of the drought tolerant transgenic rice plants was tested.

Paraquat Assay Methods:

Transgenic rice plants from ten transgenic lines were tested by paraquat assay. Tissue-cultured Zhonghua 11 plants (ZH11-TC) and empty vector transgenic plants (DP0158) were used as controls. T2 seeds were sterilized and germinated, and this assay was carried out in growth room with temperature at 28˜30° C. and humidity ˜30%. The germinated seeds were placed in a tube with a hole at the bottom, and water cultured at 30° C. for 5 days till one-leaf and one-terminal bud stage. Uniform seedlings about 3.5˜4 cm in height were selected for paraquat testing. Randomized block design was used in this experiment. There were five blocks, each of which has 16×12 holes. Each transgenic line was placed in one row (12 plants/line), and ZH11-TC and DP0158 seedlings were placed in 3 rows (3×12 plants) randomly in one block. Then the seedlings were treated with 0.8 μM paraquat solution for 7 days at 10 h day/14 h night, and the treated seedlings first encountered dark and took up the paraquat solution which was changed every two days. After treated for 7 days, the green seedlings were counted. Those seedlings that maintain green in whole without damage were considered as paraquat tolerant seedling; those with bleached leaves or stem were not considered as paraquat tolerant seedling.

Tolerance rate was used as a parameter for this trait screen, which is the percentage of plants which kept green and showed tolerant phenotype over the total plant number.

The data was analyzed at construct level (all transgenic plants compared with the control) and transgenic line level (different transgenic lines compared with the control) using a statistic model of “Y˜seg+line (seg)+rep+error”, random effect of “rep”, Statistic Method of “SAS® PROC GLIMMIX”.

### Paraquat Validation Results of OsFTR1 (DP0691) Transgenic Rice

In the first experiment, 328 of the 600 OsFTR1 transgenic seedlings (55%) kept green and showed tolerant phenotype, while 64 of the 180 (36%) seedlings from ZH11-TC showed tolerant phenotype, and 54 of the 180 (30%) DP0158 seedlings showed tolerant phenotype. The tolerance rate of all screened OsFTR1 transgenic seedlings was significantly greater than ZH11-TC and DP0158 controls at the construct level.

Further analysis at transgenic line level indicates that seven OsFTR1 transgenic lines had greater tolerance rates than ZH11-TC and DP0158 controls, and six OsFTR1 transgenic lines had significantly greater tolerance rates than ZH11-TC control and seven lines had significantly greater tolerance rates than DP0158 control (Table 4). These results demonstrate that OsFTR1 transgenic rice plants had enhanced paraquat tolerance compared to both controls of ZH11-TC and DP0158 rice plants at construct and transgenic line level at seedling stages. OsFTR1 may function in enhancing paraquat tolerance or antioxidative ability of transgenic plants.

TABLE 4
Paraquat tolerance assay of OsFTR1 transgenic rice plants (1^{st} experiment)

| | Number of | Number of | | | | | |
|---|---|---|---|---|---|---|---|
| | tolerant | total | Tolerant | CK = ZH11-TC | | CK = DP0158 | |
| Line ID | seedlings | seedlings | rate (%) | P value | P ≤ 0.05 | P value | P ≤ 0.05 |
| DP0691 | 328 | 600 | 55 | 0.0000 | Y | 0.0000 | Y |
| ZH11-TC | 64 | 180 | 36 | | | | |
| DP0158 | 54 | 180 | 30 | | | | |
| DP0691.01 | 9 | 60 | 15 | 0.0052 | | 0.0287 | |
| DP0691.02 | 20 | 60 | 33 | 0.7557 | | 0.6301 | |
| DP0691.03 | 9 | 60 | 15 | 0.0052 | | 0.0287 | |
| DP0691.04 | 39 | 60 | 65 | 0.0002 | Y | 0.0000 | Y |
| DP0691.07 | 30 | 60 | 50 | 0.0529 | | 0.0072 | Y |
| DP0691.08 | 46 | 60 | 77 | 0.0000 | Y | 0.0000 | Y |
| DP0691.11 | 46 | 60 | 77 | 0.0000 | Y | 0.0000 | Y |
| DP0691.12 | 49 | 60 | 82 | 0.0000 | Y | 0.0000 | Y |
| DP0691.13 | 48 | 60 | 80 | 0.0000 | Y | 0.0000 | Y |
| DP0691.15 | 32 | 60 | 53 | 0.0188 | Y | 0.0021 | Y |

In the second experiment, ten same OsFTR1 transgenic lines were tested. Seven days later, 362 of the 600 OsFTR1 transgenic seedlings (60%) kept green and showed tolerant phenotype, while 99 of the 180 (55%) seedlings from ZH11-TC showed tolerant phenotype, and 90 of the 180 (50%) DP0158 seedlings showed tolerant phenotype. The tolerance rate of all screened OsFTR1 transgenic seedlings was greater than ZH11-TC control, and significantly greater than DP0158 control at the construct level.

Further analysis at transgenic line level indicates that two OsFTR1 transgenic lines had significantly greater tolerance rates than ZH11-TC control and three lines had significantly greater tolerance rates than DP0158 control (Table 5). These results further demonstrate that OsFTR1 functions in enhancing paraquat tolerance or antioxidative ability of transgenic plants.

TABLE 5
Paraquat tolerance assay of OsFTR1 transgenic rice plants (2^{nd} experiment)

| | Number of | Number of | | | | | |
|---|---|---|---|---|---|---|---|
| | tolerant | total | Tolerant | CK = ZH11-TC | | CK = DP0158 | |
| Line ID | seedlings | seedlings | rate (%) | P value | P ≤ 0.05 | P value | P ≤ 0.05 |
| DP0691 | 362 | 600 | 60 | 0.1784 | | 0.0138 | Y |
| ZH11-TC | 99 | 180 | 55 | | | | |
| DP0158 | 90 | 180 | 50 | | | | |
| DP0691.01 | 35 | 60 | 58 | 0.6539 | | 0.2684 | |
| DP0691.02 | 40 | 60 | 67 | 0.1197 | | 0.0298 | Y |
| DP0691.03 | 33 | 60 | 55 | 0.9994 | | 0.5048 | |
| DP0691.04 | 45 | 60 | 75 | 0.0091 | Y | 0.0016 | Y |
| DP0691.07 | 25 | 60 | 42 | 0.0802 | | 0.2692 | |
| DP0691.08 | 33 | 60 | 55 | 0.9994 | | 0.5048 | |
| DP0691.11 | 37 | 60 | 62 | 0.3709 | | 0.1235 | |
| DP0691.12 | 44 | 60 | 73 | 0.0162 | Y | 0.0030 | Y |
| DP0691.13 | 35 | 60 | 58 | 0.6539 | | 0.2684 | |
| DP0691.15 | 35 | 60 | 58 | 0.6539 | | 0.2684 | |

### Example 5

### Generation of Transformed Maize Expressing OsFTR1

### Expression Vector for Transformation of Maize

The cDNA of OsFTR1 gene described in the Example 2 was cloned into a destination vector to get the PHP79718 vector. The vector PHP79718 contains the following expression cassettes:
1. Ubiquitin promoter::moPAT::PinII terminator; cassette expressing the PAT herbicide resistance gene used for selection during the transformation process.
2. LTP2 promoter::DS-RED2::PinII terminator; cassette expressing the DS-RED color marker gene used for seed sorting.
3. Ubiquitin promoter::OsFTR1::PinII terminator; cassette overexpressing the gene of interest, Oryza sativa OsFTR1 polypeptide.

### Example 6

### Yield Analysis of Maize Lines Expressing OsFTR1

### Method:

The transgenic plants obtained in Example 5, were used in field-based experiments to study yield enhancement and/or stability under well-watered and water-limiting conditions.

Five transgenic events were field tested in 2017 at 9 locations, drought was imposed in 6 locations by limiting irrigation at specific growth stages and three locations were well-watered. Other than irrigation treatments, the crop was managed according to local commercial practices with effective control of weeds and pests.

Yield data were collected in all locations, with 2-3 replicates per location. To evaluate the yield data, a mixed model framework was used to perform the single and multi location analysis. In the single location analysis, main effect of construct is considered as a random effect. (However, construct effect might be considered as fixed in other circumstances.) The main effect of event is considered as random. The blocking factors such as replicates and incomplete block within replicates are considered as random. In the multi-location analysis, the main effect of event or construct and its interaction with loc_id are considered as random effects. There are 3 components of spatial effects including x_adj, y_adj and autoregressive correlation as AR1*AR1 to remove the noise caused by spatial variation in the field. Yield analysis was by ASREML (VSN International Ltd) (Best Linear Unbiased Prediction) (Cullis, B. R et al (1998) Biometrics 54: 1-18, Gilmour, A. R. et al (2009). ASRemI User Guide 3.0, Gilmour, A. R., et al (1995) Biometrics 51: 1440-50).

Yield of the 5 transgenic events was compared with a bulk null control (BN). The null control comprised a physical bulk of null segregant seed from diverse events that emerged from the transformation process on the same timeline as the positive events. Seed for the events and the null was produced in the same nursery. We calculated the blup (Best Linear Unbiased Prediction) for each event and also at the construct level (combining all events). The significance test between the event and BN was performed using a p-value of 0.1 in a two-tailed test.

Yield data (bushel/acre; bu/ac) for the single-location analysis are shown in Table 6 at the level of individual event and at the aggregate construct level. The difference between the positive and null entries is also indicated. Values that differed significantly are highlighted by an asterisk, indicating a P-value less than or equal to 0.1 in a 2-tailed test. The locations are categorized according to having experienced optimum water conditions (OPT), moderate drought stress (MS), or severe drought stress (SS). Table 7 shows the data analysis across all 9 locations and also broken into environment groups based on the type of location. A generally positive effect of the transgene on yield was seen in most locations, with the exception of two MS locations. All events and the overall construct had a significant positive effect in the OPT locations. In the MS and SS locations, events 4.10 and 4.15 had positive effects, and this was significant in both environment types for event 4.15.

Effects of the transgene were evaluated for other agronomic characteristics such as plant and ear height and thermal time to shed. No effect of the transgene on these characteristics was observed (data not shown). The transgene did affect grain moisture at harvest (Table 8, 9) and test weight (Table 10, 11). The increase in moisture was positive, and reached significance in multi-location analyses (Table 9). The impact of the transgene on test weight was negative, and reached significance in those 2 locations where a negative effect on yield was observed (WO9 and WOC; Table 10). The effect of the transgene on test weight was not significant in the multi-location analyses (Table 11).

TABLE 6
Single location yield (bu/ac)

| | Drought Condition | | | | | | | | |
|---|---|---|---|---|---|---|---|---|---|
| | SS | MS | MS | MS | OPT | MS | OPT | OPT | MS |
| PHP79718 | 2.79 | −4.32 | −1.8 | * 4.06 | 7.15 | 0.74 | * 8.15 | 5.42 | 2.33 |
| (Construct) | | | | | | | | | |
| BN | 114.51 | 149.59 | 152.72 | 151.56 | 217.98 | 154.31 | 253.39 | 227.89 | 169.51 |
| EZM005.256.1.19 | −2.03 | * −12.90 | −1.77 | 2.25 | 6.5 | −0.4 | * 7.48 | 5.47 | 5.02 |
| EZM005.256.2.3 | 3.6 | * −13.79 | −1.85 | 3.34 | * 12.84 | 1.08 | 7 | 5.54 | 0.94 |
| EZM005.256.4.10 | 7.03 | 6.58 | −1.62 | 3.67 | 3.81 | 2.41 | * 8.79 | 5.45 | 2.77 |
| EZM005.256.4.11 | 1.15 | −5.79 | −1.89 | * 5.76 | 4.39 | −0.94 | * 9.06 | 5.38 | −0.69 |
| EZM005.256.4.15 | 4.2 | 4.31 | −1.9 | * 5.30 | 8.19 | 1.55 | * 8.42 | 5.27 | 3.58 |

TABLE 7
Multi-location yield (bu/ac)

| Event | 9 Iocs | OPT | MS | SS |
|---|---|---|---|---|
| PHP79718 | 1.27 | * 3.16 | 0.07 | 1.59 |
| BN | 178.23 | 235.65 | 155.74 | 118.4 |
| EZM005.256.1.19 | −0.75 | * 2.67 | −2.09 | −4.33 |
| EZM005.256.2.3 | −0.68 | * 2.70 | −2.28 | −2.82 |
| EZM005.256.4.10 | * 3.76 | * 3.68 | 2.98 | * 7.84 |
| EZM005.256.4.11 | 0.33 | * 3.22 | −1.35 | 0.07 |
| EZM005.256.4.15 | * 3.71 | * 3.57 | * 3.11 | * 7.17 |

TABLE 8
Single location moisture (%)

| | Drought Condition | | | | | | | | |
|---|---|---|---|---|---|---|---|---|---|
| | SS | MS | MS | MS | OPT | MS | OPT | OPT | MS |
| PHP79718 | 0.14 | 0.12 | 0.23 | 0.25 | 0.43 | 0.31 | −0.02 | −0.05 | −0.05 |
| (Construct) | | | | | | | | | |
| BN | 15.02 | 17.12 | 15.88 | 17.96 | 23.78 | 22.45 | 19.58 | 17.93 | 20.83 |
| EZM005.256.1.19 | 0.09 | 0.1 | 0.03 | * 0.56 | 0.33 | 0.23 | 0.05 | −0.08 | −0.07 |
| EZM005.256.2.3 | 0.19 | −0.16 | 0.29 | 0.05 | 0.39 | * 0.71 | −0.06 | −0.08 | −0.05 |
| EZM005.256.4.10 | 0.18 | 0.25 | 0.34 | 0.23 | * 1.10 | 0.32 | −0.1 | −0.02 | −0.03 |
| EZM005.256.4.11 | 0.04 | 0.25 | 0.24 | 0.19 | 0.2 | * 0.79 | 0.15 | −0.03 | −0.08 |
| EZM005.256.4.15 | 0.19 | 0.14 | 0.24 | 0.19 | 0.13 | −0.49 | −0.14 | −0.02 | −0.04 |

TABLE 9
Multi-location moisture (%)

| Event | 9 locs | OPT | MS | SS |
|---|---|---|---|---|
| PHP79718 | * 0.16 | * 0.15 | * 0.17 | 0.14 |
| (Construct) | | | | |
| BN | 18.96 | 20.42 | 18.87 | 15.03 |
| EZM005.256.1.19 | 0.1 | 0.09 | 0.1 | 0.1 |
| EZM005.256.2.3 | 0.16 | 0.11 | 0.19 | 0.15 |
| EZM005.256.4.10 | * 0.29 | * 0.33 | * 0.27 | * 0.22 |
| EZM005.256.4.11 | * 0.21 | 0.16 | * 0.26 | 0.12 |
| EZM005.256.4.15 | 0.04 | 0.05 | 0.01 | 0.12 |

TABLE 10
Single location test weight

| | Drought Condition | | | | | | |
|---|---|---|---|---|---|---|---|
| | MS | MS | OPT | MS | OPT | OPT | MS |
| PHP79718 | −0.2 | * −0.53 | * −0.33 | 0.23 | −0.04 | 0.21 | 0.02 |
| (Construct) | | | | | | | |
| BN | 56.91 | 58.02 | 57.09 | 56.47 | 56.83 | 57.67 | 57.76 |
| EZM005.256.1.19 | −0.23 | * −0.52 | * −0.60 | 0.57 | 0.01 | 0.15 | 0.01 |
| EZM005.256.2.3 | −0.23 | * −0.54 | −0.36 | 0.03 | −0.1 | 0.17 | −0.03 |
| EZM005.256.4.10 | −0.08 | * −0.50 | −0.15 | −0.16 | 0.01 | 0.3 | 0.04 |
| EZM005.256.4.11 | −0.45 | * −0.56 | * −0.47 | 0.25 | −0.08 | | −0.18 |
| EZM005.256.4.15 | −0.01 | * −0.52 | −0.06 | 0.45 | −0.04 | 0.23 | 0.25 |

TABLE 11
Multi-location test weight

| Event | 7 locs | OPT | MS |
|---|---|---|---|
| PHP79718 | −0.07 | −0.13 | −0.09 |
| (Construct) | | | |
| BN | 57.46 | 57.21 | 57.29 |
| EZM005.256.1.19 | −0.09 | −0.16 | −0.1 |
| EZM005.256.2.3 | −0.11 | −0.12 | −0.16 |
| EZM005.256.4.10 | −0.03 | −0.1 | −0.05 |
| EZM005.256.4.11 | * −0.19 | −0.08 | * −0.32 |
| EZM005.256.4.15 | 0.07 | −0.18 | 0.17 |

### Example 7

Laboratory Drought Screening of Rice Drought Tolerance Genes in Arabidopsis To understand whether rice drought tolerance genes can improve dicot plants' drought tolerance, or other traits, the rice drought tolerance gene vectors can be transformed into Arabidopsis (Columbia) using floral dip method by Agrobacterium mediated transformation procedure and transgenic plants were identified (Clough, S. T. and Bent, A. F. (1998) The Plant Journal 16, 735-743; Zhang, X. et al. (2006) Nature Protocols 1: 641-646).

Progeny of the regenerated plants, such as T1 plants, can be subjected to a soil-based drought stress. Using image analysis, plant area, volume, growth rate and color can be measured at multiple times before and during drought stress. Significant delay in wilting or leaf area reduction, a reduced yellow-color accumulation, and/or an increased growth rate during drought stress, relative to a control, will be considered evidence that the gene functions in dicot plants to enhance drought tolerance.

### Example 8

### Field Low Nitrogen Tolerance Assays of Mature Transgenic Rice Plants

### Method:

Field low nitrogen tolerance assays were carried out in Beijing. Two nitrogen levels: N-0 (using fertilizer without nitrogen) and N-1 (normal fertilizer at 180 kg Nitrogen/ha) were set in the experiments. Transgenic seeds were soaked by 25% prochloraz solution by diluting 1000 times for 36 h at room temperature, soaked in water for 12 h, and then germinated for 12 h at 35-37° C. in an incubator and refined bud for 12 h at room temperature. The germinated seeds were planted in a seedbed field. At 3-leaf stage, the seedlings were transplanted into two testing fields, with 4 replicates and 10 plants per replicate for each transgenic line, and the 4 replicates were planted in the same block. The ZH11-TC and DP0158 plants were planted nearby the transgenic lines in the same block, and were used as controls in the statistical analysis.

The rice plants were managed by normal practice using pesticides, but applying phosphorous fertilizer and potassium fertilizer for N-0 treatment and normal fertilizers for N-1.

At the end of the season, six representative plants of each transgenic line were harvested from the middle of the row per line. The grain weight per plant data were statistically analyzed using mixed linear model by ASRemI program. Positive transgenic lines are selected based on the analysis (P<0.1).

### Field NUE Validation Results of OsFTR1 (DP0691) Transgenic Rice

As shown in Table 12, the average grain yield of OsFTR1 transgenic rice (22.16 g per plant) is greater than that of ZH11-TC and significantly greater than DP0158 control under low nitrogen condition (N-0) at construct level. Four lines showed significantly greater grain yield per plant than ZH11-TC, six lines showed significantly greater grain yield per plant than DP0158 control at event level under low nitrogen conditions.

As shown in Table 13, the same twelve events were tested under field normal nitrogen conditions (N-1). The grain yield of OsFTR1 transgenic rice was 23.54 g per plant, which is greater than that of DP0158 but lower than that of ZH11-TC at construct level. Five lines showed greater grain yield per plant than ZH11-TC, and four lines showed significantly greater grain yield per plant than DP0158 control at line level under normal nitrogen condition.

These results indicate that OsFTR1 transgenic rice plants obtained greater grain yield per plant under low nitrogen condition. Over-expression of OsFTR1 may improve low nitrogen tolerance and/or NUE.

TABLE 12
Grain yield analysis of OsFTR1 transgenic rice under field low nitrogen condition (N-0)

| | Number of | Number of | | | | | | | |
|---|---|---|---|---|---|---|---|---|---|
| | survival | harvested | Yield per | CK = ZH11-TC | | | CK = DP0158 | | |
| Line ID | plants | plants | Plant (g) | Diff | P value | P ≤ 0.1 | Diff | P value | P ≤ 0.1 |
| DP0691 | | | 22.16 | 1.51 | 0.169 | | 2.58 | 0.018 | Y |
| (Construct) | | | | | | | | | |
| ZH11-TC | 40 | 24 | 20.64 | | | | | | |
| DP0158 | 40 | 24 | 19.58 | | | | | | |
| DP0691.01 | 40 | 21 | 24.15 | 3.50 | 0.022 | Y | 4.57 | 0.003 | Y |
| DP0691.02 | 40 | 23 | 21.18 | 0.54 | 0.724 | | 1.60 | 0.292 | |
| DP0691.03 | 40 | 22 | 24.66 | 4.01 | 0.009 | Y | 5.08 | 0.001 | Y |
| DP0691.04 | 40 | 20 | 19.23 | −1.41 | 0.355 | | −0.35 | 0.820 | |
| DP0691.07 | 40 | 24 | 25.61 | 4.96 | 0.001 | Y | 6.03 | 0.000 | Y |
| DP0691.08 | 40 | 24 | 24.34 | 3.70 | 0.017 | Y | 4.76 | 0.002 | Y |
| DP0691.09 | 40 | 24 | 18.68 | −1.97 | 0.196 | | −0.90 | 0.551 | |
| DP0691.10 | 40 | 18 | 22.19 | 1.55 | 0.311 | | 2.61 | 0.085 | Y |
| DP0691.11 | 40 | 24 | 22.64 | 1.99 | 0.191 | | 3.06 | 0.044 | Y |
| DP0691.12 | 40 | 24 | 21.27 | 0.63 | 0.682 | | 1.69 | 0.265 | |
| DP0691.13 | 40 | 23 | 21.37 | 0.72 | 0.636 | | 1.79 | 0.238 | |
| DP0691.15 | 40 | 23 | 20.56 | −0.08 | 0.957 | | 0.98 | 0.528 | |

TABLE 13
Grain yield analysis of OsFTR1 transgenic rice under field normal nitrogen condition (N−1)

| | Number of | Number of | | | | | | | |
|---|---|---|---|---|---|---|---|---|---|
| | survival | harvested | Yield per | CK = ZH11-TC | | | CK = DP0158 | | |
| Line ID | plants | plants | plant (g) | Diff | P value | P ≤ 0.1 | Diff | P value | P ≤ 0.1 |

| | | | | | | | | |
|---|---|---|---|---|---|---|---|---|
| DP0691 | | | 23.54 | −0.92 | 0.448 | 1.32 | 0.279 | |
| (Construct) | | | | | | | | |
| ZH11-TC | 40 | 24 | 24.46 | | | | | |
| DP0158 | 40 | 24 | 22.22 | | | | | |
| DP0691.01 | 40 | 24 | 21.77 | −2.69 | 0.123 | −0.45 | 0.795 | |
| DP0691.02 | 40 | 24 | 22.96 | −1.50 | 0.389 | 0.74 | 0.673 | |
| DP0691.03 | 40 | 24 | 26.20 | 1.74 | 0.320 | 3.98 | 0.023 | Y |
| DP0691.04 | 40 | 23 | 19.94 | −4.52 | 0.010 | −2.28 | 0.191 | |
| DP0691.07 | 40 | 23 | 25.28 | 0.82 | 0.638 | 3.06 | 0.080 | Y |
| DP0691.08 | 40 | 24 | 25.26 | 0.79 | 0.649 | 3.04 | 0.082 | Y |
| DP0691.09 | 40 | 24 | 19.91 | −4.55 | 0.009 | −2.31 | 0.186 | |
| DP0691.10 | 40 | 20 | 23.88 | −0.59 | 0.737 | 1.65 | 0.344 | |
| DP0691.11 | 40 | 24 | 25.66 | 1.20 | 0.492 | 3.44 | 0.049 | Y |
| DP0691.12 | 40 | 24 | 24.98 | 0.52 | 0.768 | 2.76 | 0.114 | |
| DP0691.13 | 40 | 24 | 23.37 | −1.10 | 0.530 | 1.14 | 0.512 | |
| DP0691.15 | 40 | 24 | 23.27 | −1.19 | 0.494 | 1.05 | 0.549 | |

1. (canceled)

2. (canceled)

3. (canceled)

4. (canceled)

5. A modified plant or seed comprising an increased expression of at least one polynucleotide encoding a FTR1 polypeptide compared to a control plant or seed, wherein the polynucleotide comprises: (a) a polynucleotide with a nucleotide sequence of at least 85% identity to SEQ ID NO: 1; (b) a polynucleotide with a nucleotide sequence of at least 85% identity to SEQ ID NO: 2; (c) a polynucleotide encoding a polypeptide comprising an amino acid sequence of at least 90% identity to SEQ ID NO: 3, wherein said plant exhibits improved drought tolerance and/or improved nitrogen stress tolerance and/or increased yield when compared to the control plant.

6. The plant of claim 5, wherein the plant comprises in its genome a recombinant DNA construct comprising a polynucleotide encoding a polypeptide comprising an amino acid sequence of at least 90% identity to SEQ ID NO: 3 operably linked to at least one regulatory element.

7. The plant of claim 5, wherein the plant comprises in its genome a targeted genetic modification at a genomic locus encoding the polypeptide, wherein the targeted genetic modification increases the expression and/or activity of the endogenous polypeptide.

8. The plant of claim 5, wherein said plant is selected from the group consisting of rice, maize, soybean, sunflower, sorghum, canola, wheat, alfalfa, cotton, barley, millet, sugar cane and switchgrass.

9. A method of increasing drought tolerance and/or increasing nitrogen stress tolerance and/or increasing yield in a plant, comprising increasing the expression of at least one polynucleotide encoding a FTR1 polypeptide in the plant compared to a control plant, wherein the polynucleotide comprises: (a) a polynucleotide with a nucleotide sequence of at least 85% identity to SEQ ID NO: 1; (b) a polynucleotide with a nucleotide sequence of at least 85% identity to SEQ ID NO: 2; and (c) a polynucleotide encoding a polypeptide comprising an amino acid sequence of at least 90% sequence identity to SEQ ID NO: 3.

10. The method of claim 9, wherein the expression of the polynucleotide is increased by:
(a) increasing the expression of the polynucleotide encoding a FTR1 polypeptide in plant by introducing a recombinant DNA construct into the plant, wherein the recombinant DNA construct comprises the polynucleotide encoding the FTR1 polypeptide operably linked to at least one heterologous regulatory element, wherein the polynucleotide encodes the polypeptide having an amino acid sequence of at least 90% identity compared to SEQ ID NO: 3; or
(b) introducing into the plant a targeted genetic modification at a genomic locus encoding the polypeptide, wherein the targeted genetic modification increases the expression and/or activity of the polypeptide.

11. (canceled)

12. (canceled)

13. (canceled)

14. (canceled)

15. The method of claim 10, wherein the targeted genetic modification is introduced using zinc finger nuclease, Transcription Activator-Like Effector Nuclease (TALEN), CRISPR-cas, guided Cas endonuclease, meganuclease or CRISPR-Cas ribonucleoprotein complexes.

16. A method of identifying one or more alleles associated with increased yield in a population of rice plants, the method comprising:
(a) detecting in a population of rice plants one or more polymorphisms in (i) a genomic region encoding a polypeptide or (ii) a regulatory region controlling expression of the polypeptide, wherein the polypeptide comprises the amino acid sequence selected from the group consisting of SEQ ID NO: 3, or a sequence that is 90% identical to SEQ ID NO: 3, wherein the one or more polymorphisms in the genomic region encoding the polypeptide or in the regulatory region controlling expression of the polypeptide is associated with yield; and
(b) identifying one or more alleles at the one or more polymorphisms that are associated with increased yield.

17. The method of claim 16, wherein the one or more alleles associated with increased yield is used for marker assisted selection of a rice plant with increased yield.

18. The method of claim 16, wherein the one or more polymorphisms is in the coding region of the polynucleotide.

19. The method of claim 16, wherein the regulatory region is a promoter.

20. The method of claim 9, wherein said plant is selected from the group consisting of rice, maize, soybean, sunflower, sorghum, canola, wheat, alfalfa, cotton, barley, millet, sugar cane and switchgrass.


SEQUENCE LISTING (5 sequences)

SEQ ID NO: 1 - DNA, 577 bp, Oryza sativa
caatcggcatctcttatcctcacgtgcgcaccacctgctcgatgaaattccccacacggccaccaaccgtgccgatggcacccgccaccgccaccgccgtggcgtccccctccacctcctcccggctcctacaccgcagcctcctctcgccaacacccacggccgctcgctgcctccggccgccgctgtgccgcgggcgcctccgcaccgttcgccaggttgtcgccaacggcgacgtctcctcgccgtcctcggacgtggccgccgaggagtccgcggcggcccccaagatcggcaagcgcgtgcgcgtcacggcgccggtacgcgtccaccacgtctccaaggcgccggacctggacatctgtggcatggagggtgtggtcaagcagtacgtcggcatctggaagggcaaacgcatcacggccaatctccccttcaaggtggagttcgagctcagggtggatgggcaggacaaaccggtccggttcttcgcccaccttcgagaggacgaattcgagctcgtcgaagacgaatagctgaataggtctcttttacagaataatgtgctctcgtcgtc

SEQ ID NO: 2 - DNA, 495 bp, Oryza sativa
atgaaattccccacacggccaccaaccgtgccgatggcacccgccaccgccaccgccgtggcgtccccctccacctcctcccggctcctacaccgcagcctcctctcgccaacacccacggccgctcgctgcctccggccgccgctgtgccgcgggcgcctccgcaccgttcgccaggttgtcgccaacggcgacgtctcctcgccgtcctcggacgtggccgccgaggagtccgcggcggcccccaagatcggcaagcgcgtgcgcgtcacggcgccggtacgcgtccaccacgtctccaaggcgccggacctggacatctgtggcatggagggtgtggtcaagcagtacgtcggcatctggaagggcaaacgcatcacggccaatctccccttcaaggtggagttcgagctcagggtggatgggcaggacaaaccggtccggttcttcgcccaccttcgagaggacgaattcgagctcgtcgaagacgaatag

SEQ ID NO: 3 - protein, 164 aa, Oryza sativa
MKFPTRPPTVPMAPATATAVASPSTSSRLLHRSLLSPTPTAARCLRPPLCRGRLRTVRQVVANGDVSSPSSDVAAEESAAAPKIGKRVRVTAPVRVHHVSKAPDLDICGMEGVVKQYVGIWKGKRITANLPFKVEFELRVDGQDKPVRFFAHLREDEFELVEDE

SEQ ID NO: 4 - DNA, 23 bp, Artificial Sequence
  Forward primer for cloning cDNA of OsFTR1 gene
caatcggcatctcttatcctcac

SEQ ID NO: 5 - DNA, 23 bp, Artificial Sequence
  Reverse primer for cloning cDNA of OsFTR1 gene
gacgacgagagcacattattctg